(19) United States
(12) Patent Application Publication
(10) Pub. No.: US 2011/0066107 A1
(43) Pub. Date: Mar. 17, 2011

(54) NEEDLE PROTECTIVE DEVICE

(76) Inventor: John Stephens, Alpharetta, GA (US)

(21) Appl. No.: 12/992,350

(22) PCT Filed: May 14, 2009
(86) PCT No.: PCT/US09/44001

§ 371 Date: Nov. 12, 2010

Related U.S. Application Data
(60) Provisional application No. 61/127,742, filed on May 14, 2008.

(51) Int. Cl. A61M 5/32; A61M 5/50

(52) U.S. Cl. 604/110; 604/198

(57) ABSTRACT

A needle protective device is provided for reducing inadvertent needle sticks. A tubular member of the needle protective device is mounted on a needle hub and extends about a needle. The tubular member is selectively axially movable between a relaxed position and a compressed position. An end cap defining a first chamber is attached to an end of the tubular member. The end cap has an opening through which a user can selectively pass the tip of the needle. In one aspect, the end cap has a locking means configured such that when the end cap is in use, as described below, the locking means pre-aligns the needle tip with the opening of the end cap to allow the needle to exit the first chamber through passive activation.

This application claims priority to and the benefit of U.S. Provisional Application No. 61/127,742, filed on May 14, 2008, which is incorporated in its entirety in this document by reference.

### FIELD OF THE INVENTION

This invention relates generally to the field of hypodermic needles, and more specifically, a protective device to reduce inadvertent needlestick incidents.

### BACKGROUND OF THE INVENTION

Infectious diseases can be transmitted to medical personnel and others by way of inadvertent needle sticks. Needlestick injuries occur frequently, most often between the time the medication is injected into the patient and the time the syringe is disposed of. Injuries occur before, during and after the clinical process. Needlestick injuries after use of the needle have been reduced with current needle protective devices, but needlestick injuries remain unaddressed during the clinical process. It is therefore desirable to provide a needle protective device to reduce the number of needlestick incidents during the complete clinical process.

### SUMMARY OF THE INVENTION

According to various embodiments, a needle protective device is provided for reducing inadvertent needle sticks. In one embodiment, the needle protective device comprises a tubular member and an end cap. The device can be activated automatically in a hygienic manner prior to the clinical procedure using conventional clinical techniques such as the forward motion of a needle towards a patient or filling vial while the hands of a user remain safely behind the tip of the needle. In one aspect, upon removal of the needle tip from a patient or filling vial, the hands of the user can remain behind the exposed tip of the needle with automatic, passive encasement of the needle tip. Thus, the automatic passive activation and automatic passive encasement of the tip of the needle can help insure the user that the safety feature is activated and remains in a protective condition throughout the entire clinical process.

In one aspect, the tubular member can be formed of resilient flexible material and can be mounted on a needle hub. The tubular member can extend about at least a portion of a needle projecting from the needle hub. In this aspect, the tubular member can be selectively axially movable between a first relaxed position and a second compressed position. When the tubular member is moved from the first relaxed position to the second compressed position, the tubular member can store resilient force.

In another aspect, the end cap can be mounted to an end of the tubular member opposite the needle hub. The tubular member can have a plurality of axial slits formed thereon that extend axially over at least a portion of the tubular member. Optionally, the axial slits can be diametrically opposed to each other and can have notches selectively formed along a portion of the edges of the axial slits, which allow portions of the tubular member to controllably bow outwardly when the tubular member is axially compressed such that the end cap is moved axially towards the needle hub. Additionally, the size of the axial slits can increase or decrease the amount of resilient force stored within a compressed tubular member.

In one aspect, the end cap can be formed from a rigid material and can define a first chamber that has an opening through which a user of the device can selectively pass the needle tip. In another aspect, the end cap can have one or multiple molded or inserted locking members that interact in a flexible, locking and/or keyed condition to allow at least a portion of a needle one-way directional access into a second chamber in order to lock the needle into the second chamber of the end cap, thus preventing the reuse of the device. In this aspect, the locking member(s) can provide tactile feedback to a user when the needle is in the process of being locked. Additionally, in this aspect, the locking member(s) can interact to support the needle in the open position while guiding the needle during automatic passive activation.

In a further aspect, a pierceable protective covering can be attached to, inserted into, or incorporated into the end cap such that the protective covering seals the opening of the end cap. In one aspect, the pierceable protective covering can comprise infection control or aseptic materials. The pierceable protective covering can be selectively pierced by the needle tip as the end cap is moved axially toward the first end of the tubular member when the tubular member is moved from the first relaxed position to the second compressed position. The pierceable protective covering can be formed from a material having a thickness configured to apply a compressive force onto the needle when pierced that is less than the resilient force stored therein the tubular member when the tubular member is moved from the first relaxed position to the second compressed position. Thus, when it is not necessary for a tip of the needle to be exposed, the resilient forces in the compressed tubular member can cause the tubular member to move axially so that the tip of the needle will reside within the first chamber in the end cap. Additionally, the pierceable protective covering can provide a tactile feedback to a user when pierced.

In one aspect, the opening in the end cap can be aligned with the tip of the needle. In another aspect, the tubular member can have a skewed end mounted on the needle hub, so that the longitudinal axis of the tubular member is not parallel with the longitudinal axis of the needle. In this aspect, the opening in the end cap can be misaligned with the tip of the needle, thereby requiring a user to move the end cap in order to pass the needle through the opening for use, or into the first chamber in the end cap for user protection.

In use, the needle protective device can help reduce the number of inadvertent needlestick injuries. In one embodiment, with the pre-alignment of the needle tip to the opening in the end cap, the device can automatically be passively activated prior to the clinical procedure by a forward or downward pressure on or by any direct contact of the end cap to a second surface, such as, for example and without limitation, the skin of a patient, under pressure. The needle protective device allows users of the device to maintain their hands behind the exposed needle tip throughout the activation and clinical procedures, with automatic, passive activation and/or encasement of the needle tip during drug filling steps, intermediary clinical steps, procedural interruptions, and the like. This passive activation can provide ease of use through direct needle contact to a patient, filling bottle or other biological surfaces.

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

In the accompanying drawings which illustrate by way of example preferred embodiments of the invention:

FIG. 1 is a side view of a needle protective device and needle assembly according to one embodiment, showing a tubular member, and an end cap.

FIG. 2 is a side cross-sectional view of the needle protective device and needle assembly of FIG. 1.

FIG. 3 is a perspective view of the tubular member of FIG. 1.

FIG. 4 is a top cross-sectional view of the end cap of FIG. 2 along line 4-4.

FIG. 5 is perspective view of an end cap, according to one embodiment.

FIG. 6A is a side cross-sectional view of a needle protective device showing a tubular member, an end cap, and a pierceable protective covering, according to one embodiment.

FIG. 6B is a bottom cross-sectional view of the needle protective device of FIG. 6A.

FIG. 7 is a top cross-sectional view of an end cap according to one embodiment.

FIG. 8 is a top cross-sectional view of an end cap according to one embodiment.

FIG. 9 is a top cross-sectional view of an end cap according to one embodiment.

FIG. 10 is a top cross-sectional view of an end cap according to one embodiment.

FIG. 11 is a side cross-sectional view of an end cap according to one embodiment.

FIG. 12 is a perspective view of an end cap according to one embodiment.

FIG. 13 is a perspective view of an end cap according to one embodiment.

FIG. 14 is a top view of an end cap and protective covering, according to one embodiment.

FIG. 15 is a side perspective view of an end cap and protective covering, according to one embodiment

FIG. 16 is a side view of a needle protective device and needle assembly according to one embodiment, showing a syringe, a tubular member, and an end cap.

FIG. 17 is a side view of a needle protective device and needle assembly according to one embodiment, showing a syringe, a tubular member, and an end cap.

FIG. 18 is side view of a needle assembly and tubular member, according to one embodiment.

FIG. 19 is side view of a needle assembly and tubular member, according to one embodiment.

FIG. 20 is side view of a needle assembly and tubular member, according to one embodiment.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “needle” can include two or more such needles unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or can not occur, and that the description includes instances where said event or circumstance occurs and instances where it does not.

As used herein, the term “needle hub” means any needle hub, retractable syringe luer slip or lock collar, or any other syringe/cylinder type hubs needle configuration, wherein a needle is projecting from a hub, retractable syringe, luer slip or lock collar or other cylinder type needle hub device.

As used herein, the term “passive activation” means any safety feature wherein a needle protective device is activated through a normal course of a clinical process use, such as, for example and without limitation, forward or downward motions of the tip or needle towards a patient during the clinical injections procedures where the hands and fingers remain behind the needle tip.

As used herein, the term “passive encasement” means any safety feature wherein a needle protective device is pre-activated before clinical use thereby providing automatic encasement of the needle tip during and after the clinical risk window, such as, for example and without limitation, during medication draw-up prior to injections, procedural interruptions due to patient instability, and throughout the continuum clinical procedure to disposal.

Reference will now be made in detail to the present preferred embodiment(s) of the invention, examples of which are illustrated in the accompanying drawings. Wherever possible, the same reference numbers are used throughout the drawings to refer to the same or like parts.

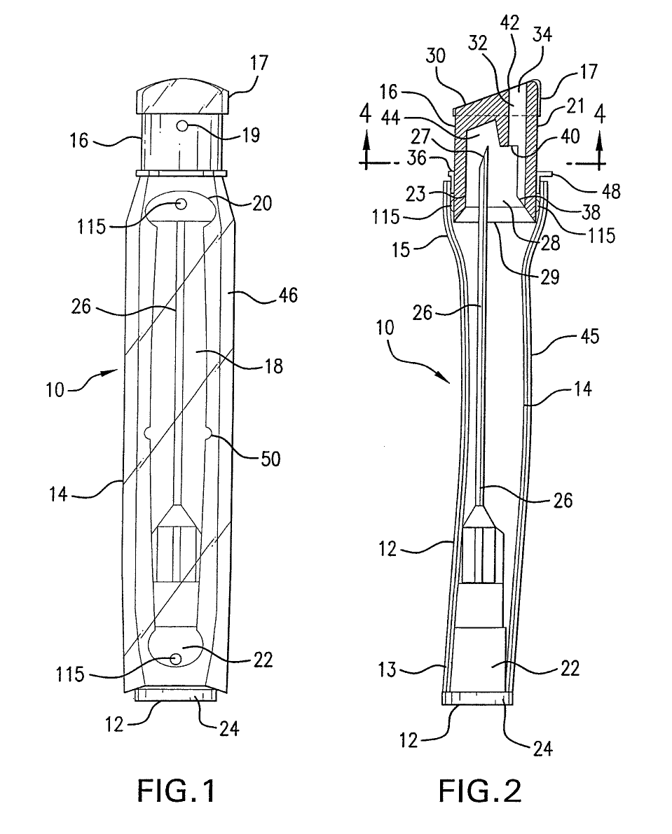

FIGS. 1 and 2 of the accompanying drawings illustrate one embodiment of a needle protective device 10 and a needle assembly 12. The needle protective device of this embodiment can comprise a tubular member 14 and an end cap 16. In another aspect, the needle protective device can further comprise a pierceable protective covering 17 comprising infection control or aseptic materials. The needle assembly can comprise a needle hub 22, an end rim 24, and an elongate needle 26 extending from the hub. In this embodiment, the tubular member of the needle protective device can be attached to the needle hub of the needle assembly.

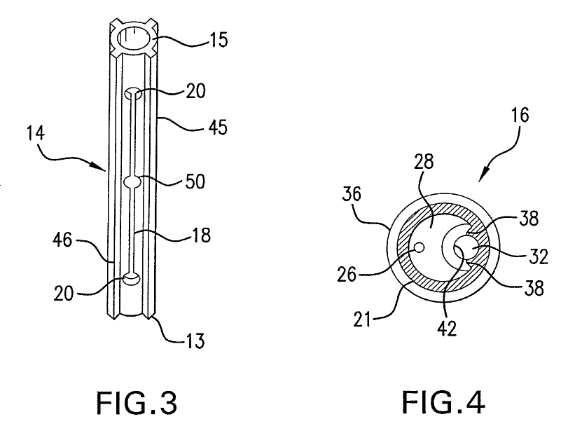

Referring to FIG. 3, in one aspect, the tubular member 14 can be cylindrical in shape having a circular cross-sectional of substantially constant diameter. It is contemplated, however that the tubular member can be other shapes and that the tubular member cross-sectional shape can also be other shapes, such as, for example, and not meant to be limiting, square, rectangular, or oval. The tubular member 14 can be formed from resilient polymeric materials, such as for example and not meant to be limiting, silicone rubber. In one aspect, the tubular member can have a Shore hardness of between approximately 30-80. In another aspect, the tubular member 14 can withstand gamma or other radiation for sterilization purposes, and can be stable up to a temperature of at least 200 degrees C. The tubular member can also be ultraviolet resistant to a substantial extent. In yet another aspect, at least a portion of the tubular member can be transparent; however, it is contemplated that at least a portion of the tubular member 14 can be color coded to indicate, for example, needle dimensions or other needle properties. It is also contemplated that tubular member can be formed by conventional manufacturing processes, such as, for example and not meant to be limiting, extrusion or injection molding.

The tubular member 14 can have a longitudinal axis, a first end 13 and a second end 15. The first end can be configured for mounting on a needle hub 22, as will be described below. The second end of the tubular member can be configured for mounting the end cap thereon, as will also be described below. In one aspect, the tubular member can be selectively axially movable between a first relaxed position and a second compressed position, by urging the second end 15 of the tubular member substantially along the longitudinal axis of the tubular member 14 towards the first end 13. In this aspect, the tubular member can store resilient force when the tubular member 14 is moved from the first relaxed position to the second compressed position.

In another aspect, the tubular member 14 can have a gripping means 45 formed thereon. In one aspect, the gripping means can consist of a flange on the tubular member at or adjacent each end. In another aspect, the gripping means can include a plurality of formations disposed about the circumference of the tubular member 14. These formations can be in various forms, such as, for example and not meant to be limiting, lugs or ribs. As illustrated in FIG. 3, the gripping means can comprise a plurality of circumferentially disposed axial ribs 46 extending over at least a portion of the length of the tubular member. The axial ribs can enable the ends of the tubular member to be gripped when these ends are stretched over the needle hub 22 and the end cap 16 during assembly, as will be more fully described below. The axial ribs 46 can be particularly useful in an automated assembly process when they can be releasably engaged with suitable mechanical devices such as clamps to facilitate the required stretching of the tubular member. In a further aspect, the axial ribs can also serve to stiffen the tubular member.

In yet another aspect, the first end 13 of the tubular member can be skewed, such that a plane in which the first end is located is not normal to the longitudinal axis of the tubular member 14. When the skewed first end of the tubular member is mounted to the needle hub 22, as will be described more fully below, the skew of the first end of the tubular member causes the longitudinal axis of the tubular member 14 to be unparallel to the longitudinal axis of the needle. In this aspect, the second end 15 of the tubular member can be straight, such that a plane in which the second end is located is normal to the longitudinal axis of the tubular member 14.

In still another aspect, the tubular member 14 can have a plurality of axial slits 18 formed thereon. The axial slits can extend axially over at least a portion of the tubular member, and the axial slits can be diametrically opposed to each other. The axial slits 18 can terminate at either end or both ends in substantially circular end notches 20, configured to reduce tearing of the axial slits which can occur as the ends of the axial slits are placed under tension when the tubular member is axially compressed. In another aspect, notches 50 can be selectively formed along the edges of the axial slits 18 in the tubular member 14. The notches 50 help ensure that when the tubular member 14 is axially compressed such that the second end 15 of the tubular member 14 is moved axially towards the first end 13 of the tubular member, portions of the tubular member will be disposed to fold or bow outwardly enabling the end cap 16 to be retracted until it encounters the needle hub 22, as will be described more fully below. In one aspect, the notches 50 can be formed at any location along the slits. In another aspect, the size of the axial slits and/or the end notches 20 and/or the notches 50 can increase or decrease the amount of resilient force stored within a compressed tubular member. For example, an increase in the width and/or length of the axial slits 18 means there can be a corresponding removal of material forming the tubular member 14, and therefore the amount of resilient force capable of being stored in a compressed tubular member can decrease. In another aspect, mating notches can be formed at any location along the slits. The mating notches, according to one aspect, can be configured to mate or attach to bosses to form a mechanical connection between the tubular member and the mating part to increase the pull force between the tubular member and the mated part.

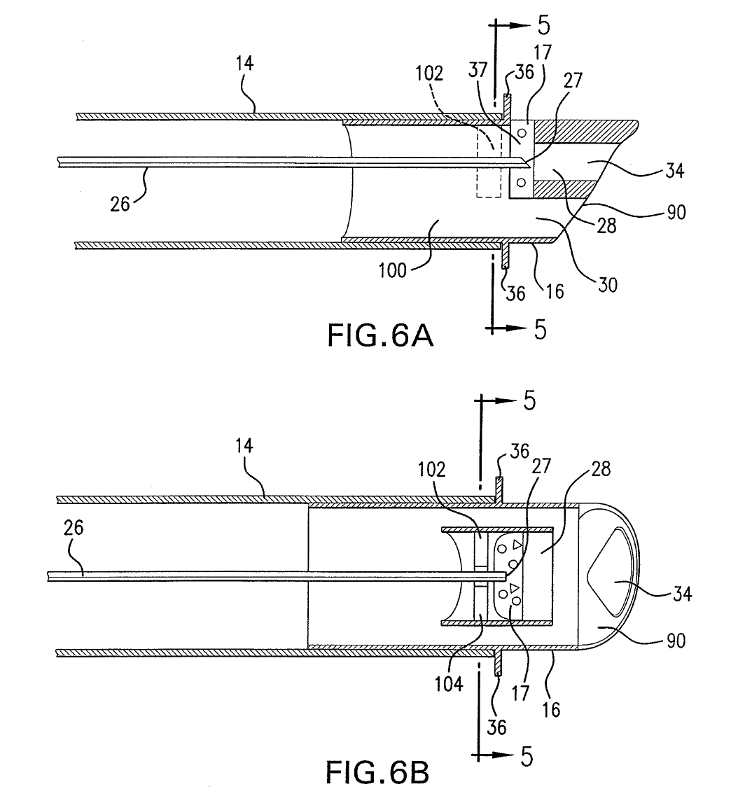

As illustrated in FIGS. 2 and 4, in one embodiment, the end cap 16 can be substantially cylindrical in shape, thereby defining a first chamber 28. The end cap 16 can formed from a relatively hard thermoplastic material, such as, for example and not meant to be limiting, polystyrene. In one aspect, at least a portion of the end cap can be transparent, so that at least a tip 27 of the needle 26 is visible, however, colored end caps are also contemplated. In one aspect, the end cap can be injection molded, though other manufacturing methods are contemplated, as are commonly known in the art. A first end 29 of the end cap can be open, and in one aspect, at least a portion of the second end 31 of the end cap 16 can be closed by a blocking surface 30. In another aspect, the blocking surface can be sloped or angled such that a user can verify the orientation of the bevel on the tip of the needle by looking only at the end cap. In still another aspect, and as illustrated in FIG. 6A, the end cap can define a slot 37 configured for receiving an infection control or aseptic material of the pierceable protective covering 17, described more fully below. In another aspect, the infection control or aseptic materials can be inserted into the slot of the end cap to provide a means to hold or gather infectious materials that come in contact with the needle cannula when in clinical use.

In one aspect, an opening 34 can be defined therein the blocking surface of the end cap that is in communication with the first chamber 28. In this aspect, a passage 32 can extend from the opening 34 in the blocking surface 30 to the first chamber. In one aspect, the opening in the blocking surface can be circular, however, the opening 34 can be other shapes, such as, for example and without limitation, D-shaped, oval, square, and the like. A wall 42 of the passage 32 can extend from the blocking surface in a direction along the longitudinal axis of the end cap, so that the wall of the passage and the blocking surface 30 define a safety chamber 44 for the tip 27 of the needle 26. In one aspect, the passage can be flared, so that a cross-sectional diameter of the passage 32 decreases as the passage progresses from the opening 34 in the blocking surface 30 towards the first end 29 of the end cap 16. The flared passage can reduce the likelihood that the tip 27 of the needle 26 can be snagged on the wall 42 of the passage and, in turn, can reduce the likelihood that the tip of the needle can thereby become damaged. In another aspect, as illustrated in FIGS. 2 and 4, the passage can have a substantially constant cross-sectional diameter.

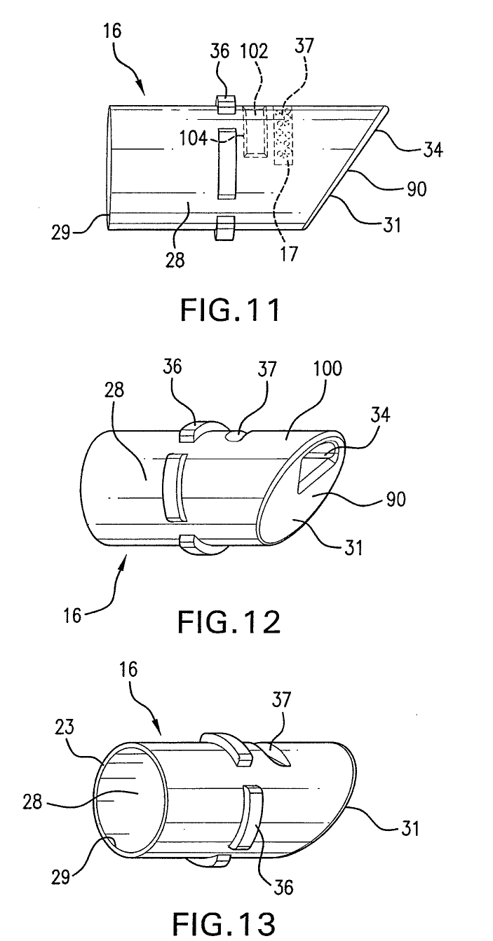

In one aspect, a flange 36 can extend around at least a portion of an outer wall 21 of the end cap. In another aspect, illustrated in FIGS. 11-13, a plurality of flanges can extend around at least a portion of the outer wall 21 of the end cap 16. In another aspect, a registration tab 48 can be formed on at least a portion of the flange 36 to assist in orientation and location of the end cap 16 in vibrating feed bowls used in an automated assembly process. The inner wall 23 of the first chamber 28 can be formed with one or more guide formations adapted to guide the tip 27 of the needle 26 to the mouth 40 of the passage 32 when the needle is displaced within the end cap, as will be described more fully below. In another aspect, the guide formations can comprise guide ribs 38 that extend in the direction of the longitudinal axis of the end cap. The guide ribs can be substantially parallel to each other. In other aspect, the guide ribs can diverge away from each other as they become closer to the first end 29 of the end cap 16 in order to facilitate guidance of the tip of the needle to the opening 34.

In another embodiment, the end cap 16 can be substantially frustoconical in shape and define a first chamber 28. In this embodiment, the first end 29 and the second end 31 of the end cap can be open. The second end of the end cap can define opening 34. The inner wall 23 of the first chamber 28 can be formed with one or more guide ribs 38. In one aspect, the guide ribs 38 can be substantially parallel to each other. In another aspect, the guide ribs can diverge rearwardly away from each other as they become closer to the first end 29 of the end cap in order to facilitate guidance of the tip of the needle. In yet another aspect, a passage 32 can extend from the opening 34 to the first chamber 28. In this embodiment with the open second end 31 of the end cap 16, when assembled as a component of the needle protection device, no alignment of the needle 26 with the opening 34 of the end cap can be necessary in order to expose the needle, as will be described more fully below. Instead, the needle may be exposed by passive activation, as will also be described more fully below.

In one aspect, the end cap 16 can be provided with a visual indicator 19, such as, for example and not meant to be limiting, a colored dot or a raised area, to provide a reference point for users of the needle protective device 10. The visual indicator can allow a user to quickly ascertain the orientation of the needle relative to the guide ribs 38 and/or opening 34 so that an injection can be administered properly.

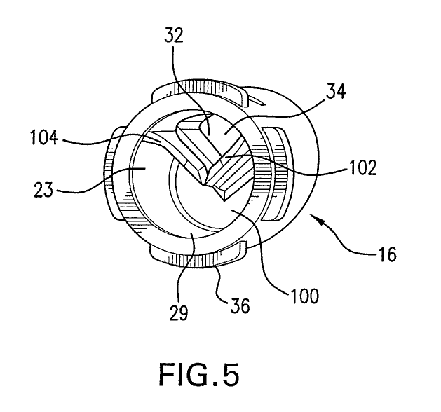

In another embodiment, and as illustrated in FIG. 5, the end cap 16 can comprise a means for selectively confining a portion of the needle to a second chamber 100 of the end cap that is not in communication with the opening 34 of the end cap. For clarity and conciseness, the means for selectively confining a portion of the needle to the second chamber of the end cap will be referred to herein as a locking means. In one aspect, the locking means can be configured such that when the end cap is in use, as described below, the locking means can pre-align the needle tip 27 with the opening 34 of the end cap 16 to allow the needle to exit the first chamber 28 through passive activation using a substantially forward movement on the device 10. In one aspect, the locking means can be at any location along the longitudinal axis of the end cap. In another aspect, the locking means can allow one-directional movement of the needle 26 so that the tip 27 of the needle can be moved adjacent a blocking surface 30 or an end dam 90, but can prevent the tip of the needle from being moved away from the blocking surface or the end dam, thereby preventing the needle 26 from exiting the first chamber 28. In order to activate the locking means, a user can exert a downward and/or a rotational motion on the end cap 16 on a surface, thereby moving the needle through the locking members 102, 104, as will be described below, into the second chamber. After activation, the locking means of the end cap can confine the needle tip 27 to the second chamber 100 of the end cap, wherein the tip 27 of the needle is prevented from exiting the second chamber 100 by the locking means in combination with an end dam 90 or blocking surface 30.

In another aspect, the locking means can provide a user of the device tactile feedback, so that the user can be aware that the needle is locked or in the process of locking without visually seeing this condition. In yet another aspect, the locking means can be configured to support the needle 26 when the needle is in an unlocked position within the end cap 16. In another aspect, the locking means can be configured to guide the needle during passive activation. In yet another aspect, the locking means can be configured to prevent movement of the needle from the first chamber to the second chamber during passive activation. In still another aspect, the locking means of the end cap can prevent the reuse of a needle after the needle 26 has been locked within the second chamber 100 of the end cap.

FIGS. 5 and 7-11 illustrate embodiments of an end cap locking means. In one embodiment, illustrated in FIG. 5, the locking means can be located at first end 29 of the end cap 16, though it is contemplated that the locking means could also be located at other positions within first chamber 28 of the end cap. The end cap can be substantially frustoconical in shape, though other shapes, such as substantially cylindrical, are also contemplated. The first end 29 of the end cap can be open, and at least a portion of the second end 31 of the end cap can be closed by end dam 90, so that opening 34 is defined therein the second end. In one aspect, a passage 32 can extend from the opening 34 to the first chamber 28.

In another aspect, illustrated in FIG. 9, a flexible locking arm 92 can have a first end 96 and a second end 98. In still another aspect, a first guide member 107 can extend from the inner wall of the first chamber transverse to the longitudinal axis of the end cap 16 through at least a portion of the first chamber 28, and a second guide member 108 can extend from the inner wall of the first chamber transverse to the longitudinal axis of the end cap 16 through at least a portion of the first chamber 28. The locking arm, first guide member, and second guide member can be formed of the same material of the end cap 16, though other materials are also contemplated. The first end of the locking arm 92 can be attached to, or alternatively formed integrally with, the inner wall 23 of the first chamber or the first guide member 107. The locking arm can normally be in a closed position, wherein the second end of the locking arm is in contact with the inner wall of the first chamber 28 or the second guide member 108. In the closed position, the locking arm 92 can define a second chamber 100. In one aspect, the locking arm can be configured such that, when the needle protective device 10 is assembled as described below, a needle can be urged against the second end 98 of the locking arm 92, thereby causing the locking arm to flex away from the inner wall of the first chamber or the second guide member momentarily, allowing the needle to enter the second chamber 100. In another aspect, the second end 98 of the locking arm can have a tab 93 configured for matingly engaging a notch formed therein the second guide member 108. In another aspect, the first and second guide members 107, 108 can support and guide the needle along the longitudinal axis of the end cap through the first chamber 28 of the end cap.

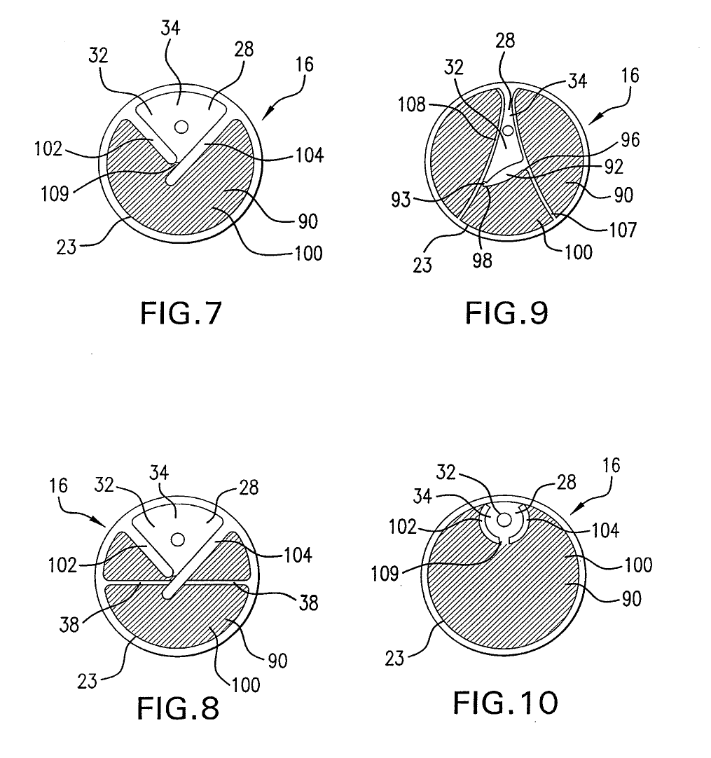

Another embodiment of an end cap locking means is illustrated in FIGS. 7 and 11. In this embodiment, the end cap 16 can be substantially frustoconical in shape, though other shapes, such as cylindrical, are also contemplated. First chamber 28 can be defined therein the end cap. The first end 29 of the end cap can be open, and at least a portion of the second end 31 of the end cap can be closed by end dam 90, so that opening 34 is defined therein the second end. In one aspect, a passage 32 can extend from the opening to the first chamber 28. A first locking member 102 and a second locking member 104 can be formed of the same material of the end cap 16, though other materials are also contemplated. The first and second locking members can be flexible, and can be attached to, or alternatively formed integrally with, the inner wall 23 of the first chamber. The first locking member 102 can extend from the inner wall of the first chamber transverse to the longitudinal axis of the end cap 16 through at least a portion of the first chamber 28. The second locking member 104 can extend from the inner wall 23 of the first chamber transverse to the longitudinal axis of the end cap so that it touches the first locking member 102 at an angle, thereby forming second chamber 100.

In this embodiment, the first locking member 102 and the second locking member 104 can interact such that, when the needle protective device 10 is assembled as described below, a needle can be urged against the second locking member 104, thereby causing the second locking member to flex away from the first locking member 102 momentarily, allowing the needle to enter the second chamber 100. Alternatively, a needle can be urged against the first locking member, thereby causing the first locking member to flex away from the second locking member momentarily, allowing the needle to enter the second chamber 100. In another aspect, and as illustrated in FIG. 8, the end cap can comprise a locking means and guide ribs 38. The guide ribs, as previously described, can support and automatically guide the tip of a needle 26 into alignment with passage 32.

Yet another embodiment of an end cap locking means is illustrated in FIG. 10. In this embodiment, the end cap 16 can be substantially frustoconical in shape, though other shapes, such as cylindrical, are also contemplated. First chamber 28 can be defined therein the end cap. The first end 29 of the end cap can be open, and at least a portion of the second end 31 of the end cap can be closed by end dam 90, so that opening 34 is defined therein the second end. In one aspect, a passage 32 can extend from the opening 34 to the first chamber 28. A first arcuate locking member 102 and a second arcuate locking member 104 can be formed of the same material of the end cap 16, though other materials are also contemplated. The first and second arcuate locking members can be attached to, or alternatively formed integrally with, the inner wall 23 of the first chamber. The first arcuate member 102 can extend from the inner wall of the first chamber transverse to the longitudinal axis of the end cap 16 through at least a portion of the first chamber 28. The second arcuate locking member 104 can extend from the inner wall 23 of the first chamber transverse to the longitudinal axis of the end cap 16 through at least a portion of the first chamber, extending towards the first arcuate member. In one aspect, a gap 109 can be formed between the first and second arcuate locking members. In another aspect, the first and second arcuate locking members can be in contact with each other, so that the gap is not present. In this embodiment, when the needle protective device 10 is assembled as described below, a needle can be urged against the first arcuate locking member 102, the second arcuate locking member 104, or both arcuate locking members, thereby causing the arcuate members to momentarily flex away from each other, allowing the needle to be easily moved past the arcuate locking members into the second chamber 100.

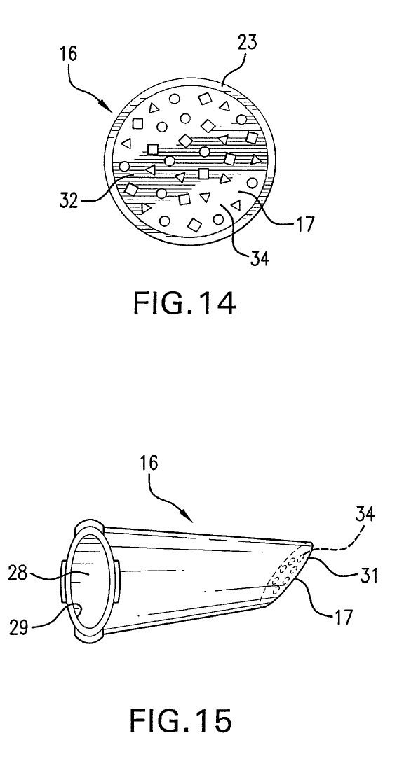

FIGS. 1, 2, 6A, 6B, 11, 14, and 15 illustrate embodiments of a pierceable protective covering 17. In various aspects, the pierceable protective covering can be attached to, inserted into or incorporated into the end cap 16 such that the protective covering seals the opening and/or the passage 32 of the end cap. In one aspect, the pierceable protective covering can comprise infection control or aseptic materials, as known in the arts. In another aspect, the protective covering can be a thin, pierceable material configured to cover the opening 34 of the end cap 16. The pierceable protective covering can be selectively pierced when, on an assembled device, as will be described more fully below, the end cap 16 is moved axially toward the first end 13 of the tubular member 14 as the tubular member is moved from the first relaxed position to the second compressed position. In one aspect, the protective covering can be formed from a material which can be impregnated with infection control or aseptic materials. In another aspect, the protective covering 17 can be formed from a fibrous material, such as for example and not meant to be limiting, paper, gauze or the like. Alternatively, in still another aspect, the protective covering can be formed from a polymeric material, such as, for example and not meant to be limiting, plastics, rubber or the like. In one aspect, as illustrated in FIGS. 1 and 2, the pierceable protective covering 17 can be configured to fit over the end cap. In another aspect, as illustrated in FIGS. 14 and 15, the pierceable protective covering can be formed within the opening 34 of the end cap 16. In still another aspect, the pierceable protective covering can be formed within the first chamber 28 of the end cap, as illustrated in FIGS. 6A, 6B, and 11. In another aspect, the pierceable protective covering can be inserted into the first chamber 28 of the end cap through the slot 37 of the end cap.

In one aspect, the combination of the materials for the protective covering 17 and the thickness of that material can be selected so that, when the protective covering is in use, the compressive force exerted onto the needle 26 by the protective covering is less than the resilient force stored therein the compressed tubular member 14 when it is in the second, compressed position. Thus, the protective covering 17 can exert a compressive, frictional force on the needle that is small enough to allow the needle protective device 10 to move freely axially under forces supplied to the needle protective device by the compressed tubular member. In another aspect, the protective covering 17 can provide a barrier that provides a user of the needle protective device a tactile feeling as the tip of the needle penetrates the protective covering. In this aspect, the user can know the approximate location of the tip 27 of the needle without visually seeing it so that the user can know that there is an exposed needle tip.

In another embodiment, the needle protective device 10 can comprise a flexible tubular member 14, an end cap 16, and a cover. In one aspect, the cover can be formed from a relatively hard thermoplastic material, such as, for example and not meant to be limiting, polystyrene. The cover can be a hollow tube having a closed end and an open end. The inner diameter of the cover can be dimensioned so that the tubular member 14 and the end cap 16 can fit therein the cover. In another aspect, the cover can have a length dimensioned to extend from the closing wall of the end cap 16 to the needle hub 22, when the device is assemble, as will be described below. In yet another aspect, the cover can be dimensioned so that the needle is selectively completely enclosed therein, thus maintaining the needle in a sterile condition.

In order to assemble the needle protective device, the second end 15 of a tubular member 14 can be frictionally engaged with the first end 29 of an end cap 16 by stretching the second end of the tubular member over the first end of the end cap. In one aspect, the end cap can have a pierceable protective covering 17 over the opening 34 of the end cap. In other aspects, the protective covering can be inserted into the opening or the slot 37 of the end cap. The protective covering can be attached to the end cap by conventional means, such as, for example and not meant to be limiting, adhesives or a friction fitting.

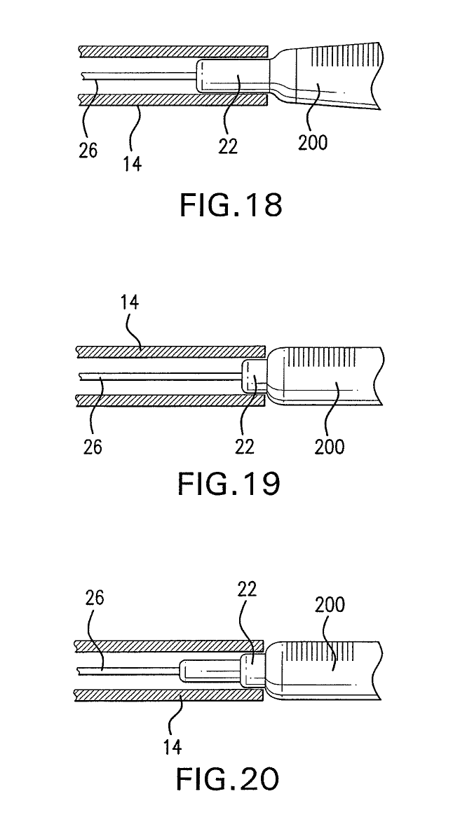

The assembled needle protective device 10 can then be inserted onto a needle assembly by stretching the first end 13 of the tubular member 14 over the needle hub 22 until the first end of the tubular member is adjacent the end rim 24. As illustrated in FIGS. 18-20, the tubular member can be stretched over a conventional needle hub, a retractable syringe, luer slip or lock collar, or any other syringe/cylinder type hub needle configuration. In one aspect, the needle hub 22 can be attached to a syringe 200, which can be a retractable syringe with a needle preassembled.

In one aspect, if the first end 13 of the tubular member is skewed, after assembly on the needle protective device onto the needle hub, the longitudinal axis of the tubular member 14 can be unparallel to the longitudinal axis of the needle 26. With reference to FIG. 2, in this aspect, the tip 27 of the needle can be disposed to lie adjacent the blocking surface 30 of the first chamber 28 opposite to the location of the passage 32. If a cover is to be included, the open end of the cover can be inserted over the end cap and tubular member until it contacts the needle hub.

In another aspect, the tubular member 14 can be mounted on the needle hub 22 in such a way that a bevel on the tip of the needle 26 slopes in the opposite direction as the blocking surface 30 of the end cap. Thus, by simply viewing the blocking surface and/or the visual indicator 19, the user can know that the bevel of the needle is in the correct disposition relative to a patient's skin. In this aspect, it is not necessary for the user to visually inspect the tip 27 of the needle itself to ensure this result.

In use, the needle assembly and thus, the needle protective device 10 can be mounted onto a syringe. In order to administer an injection, if a cover is present, the user can remove it to expose the end cap 16. If the tip 27 of the needle 26 is pre-aligned with the opening 34 in the second end 31 of the end cap, no alignment by the user is necessary and passive activation of the needle protective device 10 by the user can occur. If the needle is not pre-aligned with the opening 34 in the second end of the end cap, the needle 26 can be substantially co-axially aligned with the passage 32 and the opening 34 in the second end 31 of the end cap. If the end cap has a blocking surface 30 or a end dam 90 the end cap can be moved so that the end cap 16 is displaced sideways until the needle 26 is brought into contact with one of the guide ribs 38 which can automatically guide the tip of the needle into alignment with passage 32. If the end cap does not have guide ribs, the end cap 16 can be moved until the tip 27 of the needle 26 is aligned with passage 32. In another aspect, if the needle is not pre-aligned with the opening 34 in the second end of the end cap and if the end cap has a visual indicator 19, the end cap can be moved until the tip of the needle is aligned with passage by referring to the visual indicator. If the end cap does not have a blocking surface 30 or an end dam 90, the tip of the needle can be aligned with the passage without being moved by the user.

After alignment of the needle with the passage 32 and/or the opening 34 of the end cap, the tubular member 14 can then be axially compressed by the user to urge the second end 15 of the tubular member towards the first end 13 of the tubular member, with the center portion of the tubular member bowing outwardly. As one will appreciate, as the tubular member 14 is axially compressed, the tip 27 of the needle slides through the passage and the opening of the end cap 16. If a protective covering 17 is present, the needle will pierce the protective covering, which can provide the user a tactile feeling so that the location of the tip of the needle 26 is known. With the needle tip thus exposed, the tip 27 of the needle can be inserted into a patient, filling bottle, or a biological surface, and the tubular member 14 can be released. Resilient forces present in the axially compressed tubular member cause the second end 15 of the tubular member to slide forward and move away from the first end 13 of the tubular member axially, until the blocking surface 30 lies against the patient, filling bottle, or biological surface.

At the end of the injection procedure, as the needle 26 is withdrawn from the patient, filling bottle, or other biological surface, resilient forces present in the axially compressed tubular member 14 cause the tubular member to expand axially such that the second end 15 of the tubular member slides forward until the passage 32 in the end cap is extended over the needle 26 and the tip 27 of the needle is located in the first chamber 28, thereby reducing the likelihood that a user can receive an inadvertent needlestick. In one embodiment, if the end cap 16 has a locking means, as illustrated in FIGS. 10-13, after use of the needle is complete and with the needle located within first chamber 28, the user can laterally move and/or rotate the end cap relative to the needle 26 until the needle is confined to second chamber 100. In this embodiment, when the needle is in the second chamber, the tip 27 of the needle is opposed to the end dam 90, and thereby prevented from being extracted from the end cap, thus reducing the likelihood of accidental needle sticks.

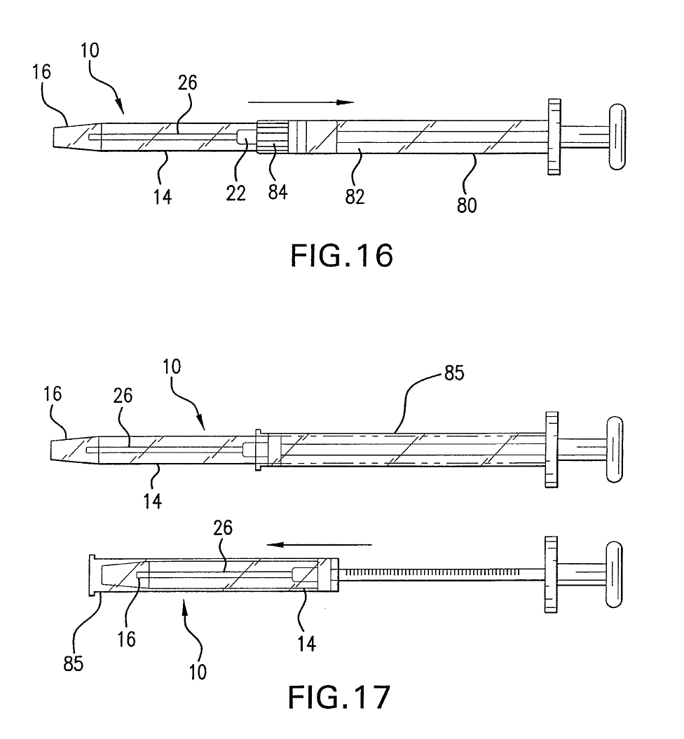

In another embodiment, as illustrated in FIG. 16, the needle protective device 10 can comprise a syringe 80, a tubular member 14, and an end cap 16. In one aspect, the syringe can be a conventional retractable polymeric syringe comprising a generally tubular chamber 82. The syringe can have threads 84 configured for selective, releasable attachment to a needle hub 22. A needle 26 can be coupled to and project outwardly from the needle hub. As described above, a tubular member can extend about at least a portion of the needle, and an end cap 16 can be mounted onto the tubular member 14. In one aspect, the tubular member can be dimensioned so that the tubular member 14, the end cap, and a protective covering 17, if present, can be retracted into the chamber 82 of the syringe as the needle assembly is retracted into the chamber of the syringe. In this aspect, the needle can be locked inside the chamber of the syringe to prevent reuse.

In another embodiment, as illustrated in FIG. 17, the needle protective device 10 can comprise a conventional 80, a tubular member 14, and an end cap 16. In one aspect, the syringe can be a conventional syringe retrofitted with a sliding barrel shield 85. In this embodiment, the tubular member 14, needle 26, and end capl6 can be dimensioned for encasement within the sliding barrel. The needle protective device can provide passive needle encasement protection during the clinical process, while following the clinical procedure the sliding barrel shield 85 can be locked over the needle protective device preventing the reuse of the needle protective device 10 and safety needle assembly.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is therefore understood that the invention is not limited to the specific embodiments disclosed herein, and that many modifications and other embodiments of the invention are intended to be included within the scope of the invention. Moreover, although specific terms are employed herein, they are used only in a generic and descriptive sense, and not for the purposes of limiting the described invention.

What is claimed is:

1. A needle protective device for a needle assembly having a needle hub and a needle projecting from the hub, the needle protective device comprising:
a tubular member of resilient flexible material which extends about at least a portion of the needle, the tubular member having a longitudinal axis, a first end and a second end, wherein the first end of the tubular member is mounted on the needle hub, wherein the tubular member is selectively axially movable between a first relaxed position and a second compressed position, and wherein the tubular member stores resilient force when the tubular member is moved from the first relaxed position to the second compressed position; and
an end cap having a longitudinal axis, a first end and a second end, wherein the first end of the end cap is fixedly mounted on the second end of the tubular member, wherein the end cap defines a first chamber for protecting a tip of the needle, and wherein the second end of the end cap defines an opening in communication with the first chamber through which a user of the device can selectively pass the tip of the needle.

2. The needle protective device of claim 1, wherein the tip of the needle is substantially co-axially aligned with the opening of the end cap and wherein the needle protective device is passively activated prior to a clinical procedure by a forward movement of the needle protective device against a surface.

3. The needle protective device of claim 1, wherein a plurality of slits are defined in a wall of the tubular member, the slits extending in an axial direction between the first and second ends of the tubular member.

4. The needle protective device of claim 3, wherein notches are formed in portions of the edges of the slits to predispose portions of the tubular member to bow outwardly when the first and second ends of the tubular member are urged together in the axial direction.

5. The needle protective device of claim 1, further comprising a pierceable protective covering configured to seal the opening of the end cap.

6. The needle protective device of claim 5, wherein the pierceable protective covering comprises an infection control material.

7. The needle protective device of claim 5, wherein the pierceable protective covering comprises an aseptic material.

8. The needle protective device of claim 5, wherein the pierceable protective covering is configured to apply a compressive force onto the needle when pierced that is less than the resilient force stored therein the tubular member when the tubular member is moved from the first relaxed position to the second compressed position.

9. The needle protective device of claim 5, wherein the pierceable protective covering is configured to provide a tactile feedback to a user when the pierceable protective covering is pierced.

10. The needle protective device of claim 5, wherein the pierceable protective covering is formed from a fibrous material.

11. The needle protective device of claim 5, wherein the pierceable protective covering is formed from a thermoplastic material.

12. The needle protective device of claim 5, wherein the pierceable protective covering is formed from a rubber material.

13. The needle protective device of claim 1, further comprising a cover configured to selectively completely enclose the needle therein.

14. The needle protective device of claim 1, wherein the end cap further comprises a second chamber, and a means for selectively confining at least a portion of the needle to the second chamber of the end cap.

15. The needle protective device of claim 14, wherein the means for selectively confining the needle to the second chamber of the end cap comprises at least one flexible locking arm configured to selectively permit the needle to be moved from the first chamber to the second chamber of the end cap and prevent the needle from being removed from the second chamber of the end cap.

16. The needle protective device of claim 15, wherein the needle is moved to the second chamber by a force substantially transverse to a longitudinal axis of the end cap.

17. The needle protective device of claim 14, wherein the means for selectively confining the needle to the second chamber of the end cap provides a tactile feedback to a user when the needle is moved into the second chamber.

18. The needle protective device of claim 14, wherein the means for selectively confining the needle to the second chamber of the end cap is configured to prevent movement of the needle from the first chamber to the second chamber during passive activation.

19. The needle protective device of claim 14, wherein the means for selectively confining the needle to the second chamber of the end cap is configured to prevent the reuse of the needle protective device.

20. The needle protective device of claim 1, wherein the first end of the tubular member is beveled such that, when mounted on the needle hub, the longitudinal axis of the tubular member is not parallel to a longitudinal axis of the needle.

21. The needle protective device of claim 20, wherein the second end of the end cap has a blocking surface opposed to the tip of the needle, and where the end cap defines a passage in communication with the first chamber that is coupled to the opening in the second end of the end cap.

22. The needle protective device of claim 21, wherein the tip of the needle is beveled and the blocking surface slopes in the opposed direction as the bevel.

23. The needle protective device of claim 1, wherein the end cap plug comprises a tubular needle cover configured to be inserted through the opening in the end cap and over a substantial length of the needle.

24. The needle protective device of claim 1, wherein the tubular member is provided with a visual indicator.

25. The needle protective device of claim 1, further comprising at least one gripping means on an exterior surface of the tubular member.

26. The needle protective device of claim 25, wherein the gripping means comprises a plurality of lugs disposed about a circumference of the tubular member.

27. The needle protective device of claim 25, wherein the gripping means comprises one or more ribs extending along an outer surface of the tubular member.

28. The needle protective device of claim 1, wherein at least a portion of the end cap is formed of transparent material.

29. The needle protective device of claim 1, wherein the end cap is provided with a visual indicator.

30. The needle protective device of claim 31, wherein the visual indicator is a colored dot.

31. The needle protective device of claim 1, wherein the end cap comprises one or more formations configured to guide the tip of the needle towards a mouth of the passage when the end of the needle is displaced sideways toward the passage.

32. The needle protective device of claim 31, wherein the passage flares outwardly from the opening of the end cap towards the mouth of the passage.

33. The needle protective device of claim 31, wherein the passage is offset to one side of the end cap.

34. The needle protective device of claim 1, wherein an external flange is provided on the end cap.

35. The needle protective device of claim 1, wherein a registration tab is provided on the end cap and protrudes laterally therefrom.

36. A needle protective device for a needle assembly having a needle hub and a needle projecting from the hub, the needle protective device comprising:
a syringe comprising:
a hollow barrel having an inner diameter;
an end wall closing the barrel at a forward end of the syringe;
an open rear end of the syringe;
a piston means in reciprocable sealing engagement with the interior of the barrel defining a first chamber in said barrel for selectively containing fluid;
a needle hub mounted on the end wall defining an interior passage; and
an aperture in the end wall communicating the interior passage of the needle hub with the first chamber;
a needle coupled to and projecting outwardly from the needle hub;
a tubular member of resilient flexible material which extends about at least a portion of the needle, the tubular member having a longitudinal axis, a first end and a second end, wherein the first end of the tubular member is fixedly mounted on the needle hub, wherein the tubular member is selectively axially movable between a first relaxed position and a second compressed position, and wherein the tubular member stores resilient force when the tubular member is moved from the first relaxed position to the second compressed position;
an end cap having a longitudinal axis, a first end and a second end, wherein the first end of the end cap is fixedly mounted on the second end of the tubular member, wherein the end cap defines a first chamber for protecting a tip of the needle, and wherein the second end of the end cap defines an opening in communication with the first chamber through which a user of the device can selectively pass an end of the needle;
wherein the tubular member is dimensioned so that the tubular member can be retracted along its longitudinal axis into the first chamber of the syringe.

37. A needle protective device for a needle assembly having a needle hub and a needle projecting from the hub, the needle protective device comprising:
a syringe comprising:
a hollow barrel having an inner diameter;
an end wall closing the barrel at a forward end of the syringe;
an open rear end of the syringe;
a piston means in reciprocable sealing engagement with the interior of the barrel defining a first chamber in said barrel for selectively containing fluid;
a needle hub mounted on the end wall defining an interior passage;
a sliding barrel shield; and
an aperture in the end wall communicating the interior passage of the needle hub with the first chamber;
a needle coupled to and projecting outwardly from the needle hub;
a tubular member of resilient flexible material which extends about at least a portion of the needle, the tubular member having a longitudinal axis, a first end and a second end, wherein the first end of the tubular member is fixedly mounted on the needle hub, wherein the tubular member is selectively axially movable between a first relaxed position and a second compressed position, and wherein the tubular member stores resilient force when the tubular member is moved from the first relaxed position to the second compressed position; and
an end cap having a longitudinal axis, a first end and a second end, wherein the first end of the end cap is fixedly mounted on the second end of the tubular member, wherein the end cap defines a first chamber for protecting a tip of the needle, and wherein the second end of the end cap defines an opening in communication with the first chamber through which a user of the device can selectively pass an end of the needle;
wherein the tubular member is dimensioned so that the tubular member can be retracted along its longitudinal axis into the sliding barrel shield of the syringe.
